(19) United States
(12) Patent Application Publication
(10) Pub. No.: US 2012/0165887 A1
(43) Pub. Date: Jun. 28, 2012

(54) IMPLANTABLE CLOSE-LOOP MICROSTIMULATION DEVICE

(76) Inventors: Shuenn-Yuh Lee, Min-Hsiung (TW); Chih-Jen Cheng, Min-Hsiung (TW); Mario Yucheng Su, Min-Hsiung (TW)

(21) Appl. No.: 13/030,830

(22) Filed: Feb. 18, 2011

(30) Foreign Application Priority Data
Dec. 24, 2010 (TW) 099145783

(51) Int. Cl. A61N 1/365

(52) U.S. Cl. 607/17

(57) ABSTRACT

An implantable closed-loop micro-stimulation device, comprising: a wireless receiver, a wireless energy conversion and storage interface, a demodulator circuit, a modulator, a main controller, a front end sensor, and a stimulation generator. The wireless energy conversion and storage interface receives AC signal through the wireless receiver, and converts it into DC voltage to charge the battery and provide a stable operation voltage. The demodulator circuit receives a wireless control signal through the wireless receiver, and demodulates it into control data and a control clock, and outputs them to the main controller. When the main controller determines that the control data is correct, the main controller outputs the stimulation parameters to the front end sensor and the stimulation generator based on the control data and the control clock, so that the stimulation generator generates a stimulation pulse signal for applying it onto the stimulation object.

### BACKGROUND OF THE INVENTION

1. Field of the Invention

The present invention relates to a micro-stimulation device, and in particular to an implantable closed-loop micro-stimulation device.

2. The Prior Arts

In recent years, various wireless coupling technologies are used extensively in the implantable biomedical electronic micro-stimulation system, and most of them adopt near-field coil coupling technique to transfer energy and data to an electronic system in a human body. The implanted electronic system operates in different operation modes depending on its application. For example, cardiac pacemaker is required to make instant response to heart beat pulses and cardiac signals, so as to save the life of a patient in time.

For a conventional cardiac pacemaker, the magnitude of stimulation pulse is several times greater than operation voltage of the circuit, such that in case it is realized through a wireless coupling system, it would require significantly large transmission power and voltage, thus it is liable to cause considerable loss of transmission energy. Moreover, for an implantable system, the area occupied by the circuit must be taken into consideration. Usually, for a conventional implantable micro-stimulation system, in order to record and keep physiological parameters and safeguard safety of human body, wireless coupling power supply has to be sacrificed, such that the unrechargeable battery utilized for power supply will make volume of the implantable micro-stimulation system overly large. In addition, usually, when external signal data is transmitted into a human body, lots of unexpected factors are liable to lead to data errors, however, for the implantable electronic micro-stimulation system presently available, the error detection mechanism is lacking, thus there is no way to ensure that the data received by the system is correct. Therefore, presently, the design and performance of the conventional wireless implantable electronic micro-stimulation system is not quite satisfactory, especially for a cardiac pacemaker, and it has much room for improvement.

### SUMMARY OF THE INVENTION

Therefore, in order to solve the problem and shortcomings of the prior art, the present invention provides an implantable closed-loop micro-stimulation device capable of transmitting energy and charging battery wirelessly, so that a medical diagnosis staff may regulate the stimulation parameters through wireless transmission. Also the service life of the power storage device can be lengthened through electricity charging, hereby reducing the painful surgery operations required for replacing the power storage device in the body of a patient, and increasing the safety and stability of the implantable micro-stimulation system in solving the problem of the prior art.

A major objective of the present invention is to provide an implantable closed-loop micro-stimulation device, wherein, a main controller provides a termination code and an error code detection for the external transmission data, to be used by a detection mechanism, thus achieving the functions of transmission data error detection and protection.

Another objective of the present invention is to provide an implantable closed-loop micro-stimulation device, wherein, a successive approximation controller, a digital-to-analog converter, and a comparator are used to form an analog-to-digital converter capable of real-time detection and real-time analog-to-digital conversion functions, so as to reduce the area occupied by two sets of comparators and digital-to-analog converters of the prior art.

A yet another objective of the present invention is to provide an implantable closed-loop micro-stimulation device, wherein, a wireless energy conversion and storage interface having a power storage device is provided, hereby avoiding the inconvenience of replacing batteries and ensuring continued operations of the device as a whole.

In order to achieve the above mentioned objective, the present invention provides an implantable closed-loop micro-stimulation device, comprising: a wireless receiver, used to receive wireless control signals; a demodulator circuit, connected to the wireless receiver to receive the wireless control signal, and demodulate it into control data and a control clock; a main controller, connected to the demodulator, and is used to receive the control data and the control clock, and detect a termination code and an error code of the control data based on preset termination detection value and error detection value, in determining correctness of the control data, and if the control data is correct, it generates a plurality of stimulation parameters based on the control data and the control clock; a front end sensor, connected to the main controller and the stimulated object, and is used to receive the stimulation parameters in generating a sensing threshold value, the front end sensor receives physiological signals transmitted from the stimulated object and compares them with the sensing threshold value and then outputs the comparison results; a modulator, connected to the front end sensor, modulates the output signal of the front end sensor and transmits the modulated signal to the external device on the out-of body; a stimulation generator, connected to the front end sensor and the main controller and is in contact with the stimulated object, the stimulation generator operates in synchronism with the main controller, and it outputs an access signal to the main controller, for the main controller to generate the stimulation parameters, such that the stimulation generator receives the stimulation parameters and the comparison results, and when the physiological signals is less than the sensing threshold values, it generates a stimulation pulse signal based on the stimulation parameters, to be applied on the stimulated objects; and a wireless energy conversion and storage interface, connected to the components mentioned above, and is used to receive the wireless control signal and convert it into signal of DC voltage for charging a power storage device, which outputs an operation voltage through a voltage stabilizer for use by the components mentioned above.

The front end sensor is composed of a first amplifier, a second amplifier, a filter, a successive approximation controller, a digital-to-analog converter, and a comparator. Wherein, the successive approximation controller, a digital-to-analog converter, and comparator can not only detect if the physiological signal is greater than the sensing threshold value, but it can also convert the sampled signals into recordable digital codes.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from this detailed descriptions.

### BRIEF DESCRIPTION OF THE DRAWINGS

The related drawings in connection with the detailed descriptions of the present invention to be made later are described briefly as follows, in which:

FIG. 1 is a block diagram of an implantable closed-loop micro-stimulation device according to the present invention;

FIG. 2 is a block diagram of a wireless energy conversion and storage interface according to the present invention;

FIG. 3 is a block diagram of a demodulator circuit according to the present invention;

FIG. 4 is a block diagram of a stimulation amplitude controller according to the present invention;

FIG. 5 is a block diagram of a front end sensor according to the present invention;

FIG. 6 is a waveform diagram of a converted clock pulse according to the present invention; and

FIG. 7 relates to flowcharts of the steps of operations of a main controller and a stimulation sequence controller respectively according to the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

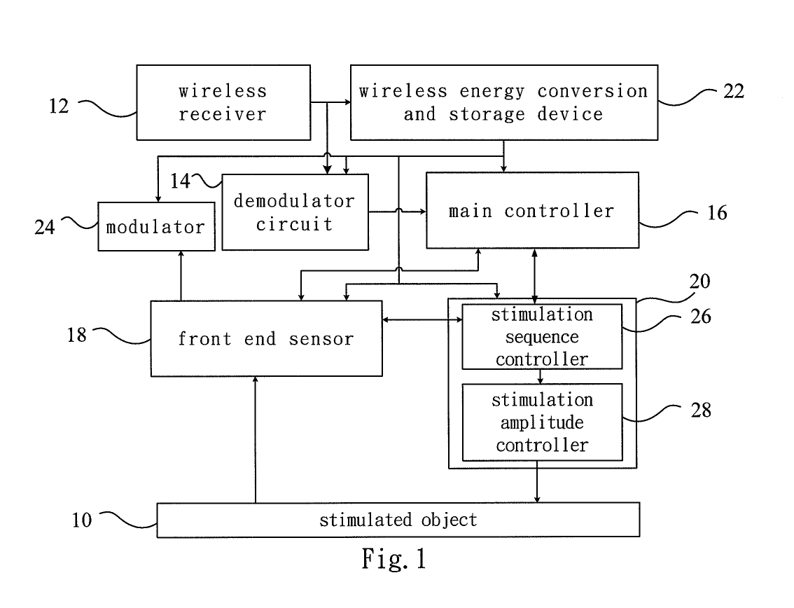

Refer to FIG. 1 for a block diagram of an implantable closed-loop micro-stimulation device according to the present invention. As shown in FIG. 1, the implantable closed-loop micro-stimulation device is in contact with a stimulated object 10 directly or through a conduction interface, comprising a wireless receiver 12, used to receive a wireless control signal, and is connected to a demodulator circuit 14; a demodulator circuit 14, used to receive the wireless control signal through the wireless receiver 12, and demodulate it into control data and a control clock, and is connected to a main controller 16; a main controller 16, provided with a set of synchronization values, a termination detection value, and an error detection value, and receives the control data and the control clock, since after demodulation the control data is a series of digital codes, therefore, the main controller 16 determines if the digital codes of the control data are the same as the set of synchronization values as based on the set of synchronization values, and in case it is, then the control data is considered in synchronism with the set of synchronization values, then the main controller detects the termination code and error code of the control data based on the termination detection value and error detection value, in determining if the control data is correct, and when it is correct, the main controller 16 generates a plurality of stimulation parameters based on the control data and control clock; a front end sensor 18, connected to the main controller 16 and the stimulated object 10, and it receives the stimulation parameters to set a sensing threshold value, such that the front end sensor 18 receives the physiological signals from the stimulated object 10, such as heart beat signals and various nervous signals, and compares them with the sensing threshold value, and then outputs the results of comparison to the main controller 16, for which to determine whether to proceed with stimulation; and a modulator 24, connected to the front end sensor 18, which receives the physiological signals and then converts them into recordable digital codes for transmitting them into the modulator 24, which outputs the digital codes after modulation, so that the user may understand clearly his physiological conditions.

The front end sensor 18 and the main controller 16 are connected to a stimulation generator 20, which is in contact with the stimulated object 10, and is operated in synchronism with the main controller 16. The stimulation generator 20 outputs an access signal to the main controller 16, for which to generate stimulation parameters, and then the stimulation generator 20 receives the stimulation parameters and the results of comparison, and when the physiological signal is less than the sensing threshold value, it generates a stimulation pulse signal based on the stimulation parameters for applying onto the stimulated object 10.

The demodulator circuit 14, the modulator 24, the main controller 16, the front end sensor 18, and the stimulation generator 20 mentioned above all require power to carry on their operations. For this reason a wireless energy conversion and storage interface 22 is provided and is connected to the wireless receiver 12, the demodulator circuit 14, the modulator 24, the main controller 16, the front end sensor 18, and the stimulation generator 20, such that it receives the wireless control signal and converts it into an operation voltage through the power management of the internal charging device and voltage stabilizer, then provides the operation voltage to the demodulator circuit 14, the modulator 24, the main controller 16, the front end sensor 18, and the stimulation generator 20 to perform various functions as required.

In the following, detailed descriptions of structures are given respectively for the wireless energy conversion and storage interface 22, the demodulator circuit 14, the stimulation generator 20, and the front end sensor 18.

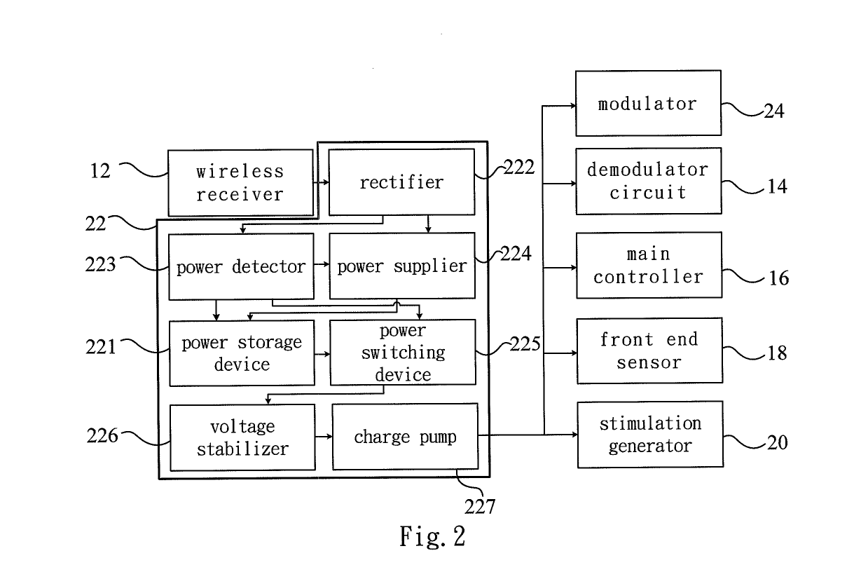

Firstly, the wireless energy conversion and storage interface 22 is described. Refer to FIG. 2 for a block diagram of a wireless energy conversion and storage interface according to the present invention. As shown in FIG. 2, the wireless energy conversion and storage interface 22 comprises a power storage device 221 and a rectifier 222. The rectifier 222 is connected to the wireless receiver 12 for receiving the wireless control signal and rectifying it into a direct current (DC) voltage. The rectifier 222 is also connected to a power storage device 221 through a power detector 223, such that the power detector 223 presets a power detection value, and receives the DC voltage for detecting the power of the power storage device 221, and when the power is equal to or greater than the power detection value, it outputs a power supply signal, and when the power is less than the power detection value, it outputs a power storage signal. The rectifier 222 and power detector 223 are connected to a power supplier 224, which receives the power storage signal and the DC voltage to charge the power storage device 221 in current. The power detector 223 and power storage device 221 are connected to a power switching device 225, which receives a power supply signal or a power storage signal to selectively turn-on or turn-off the power output channel of the power storage device 221. The power switching device 225 is connected to a voltage stabilizer 226, which receives power output by the power storage device 221 through the power output channel, and converts it into a stabilized voltage. The voltage stabilizer 226 is connected to a demodulator circuit 14, the modulator 24, the main controller 16, the front end sensor 18, and the stimulation generator 20 through a charge pump 227, such that the charge pump 227 receives the stabilized voltage and converts it into an operation voltage for supplying it to the demodulator circuit 14, modulator 24, the main controller 16, the front end sensor 18, and the stimulation generator 20 as required. In other words, through the charging technology of the present invention, the service life of the power storage device 221 can be prolonged, hereby reducing the painful surgery operations required for replacing the power storage device in the body of a patient, and raising the safety and stability of the implantable closed-loop micro-stimulation device.

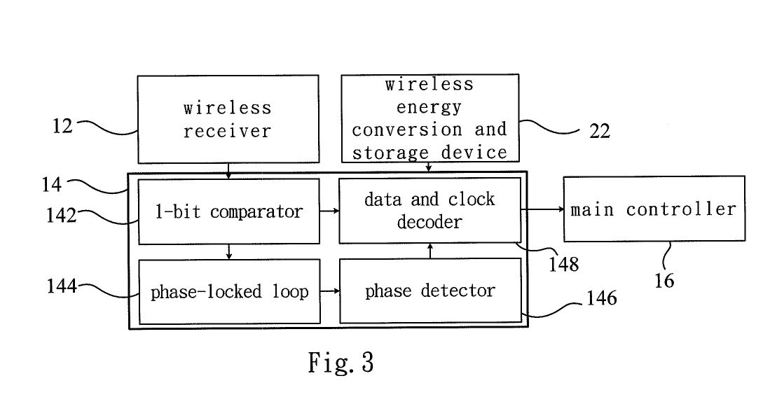

Then, refer to FIG. 3 for a block diagram of a demodulator circuit according to the present invention. As shown in FIG. 3, the demodulator circuit 14 further includes a 1-bit comparator 142, connected to the wireless receiver 12 and receives the wireless control signal, and then quantifies it into a square wave signal. The 1-bit comparator 142 is connected to a phase-locked loop 144, which receives the square wave signal and uses it to output a delay signal. The phase-locked loop 144 is connected to a phase detector 146, which receives the delay signal and uses it to determine the phase of the square wave signal, and then it generates a result signal based on the phase. The phase detector 146, the 1-bit comparator 142, and the main controller 16 are all connected to a data and clock decoder 148, which receives the result signal and the square wave signal for demodulating them into control data and a control clock.

Subsequently, refer again to FIG. 1 for describing the stimulation generator 20 as follows. The stimulation generator 20 includes a stimulation sequence controller 26 and a stimulation amplitude controller 28 connected to each other. The stimulation sequence controller 26 is connected to the front end sensor 18 and the main controller 16, and the stimulation sequence controller 26 is operated in synchronism with the main controller 16, and outputs an access signal to the main controller 16, for which to generate stimulation parameters. Then, the stimulation sequence controller 26 receives the stimulation parameters and outputs of the front end sensor 18, and sets a stimulation time and a stimulation period based on the stimulation parameters, and when the physiological signal is smaller than the sensing threshold value, it generates a stimulation clock pulse signal having stimulation time and stimulation period. The stimulation amplitude controller 28 is in contact with the stimulated object 10, and it receives the stimulation clock pulse signal and stimulation parameters, and it sets a stimulation amplitude based on the stimulation parameters, and then it outputs a stimulation pulse wave signal having the stimulation time, stimulation period, and stimulation amplitude mentioned above.

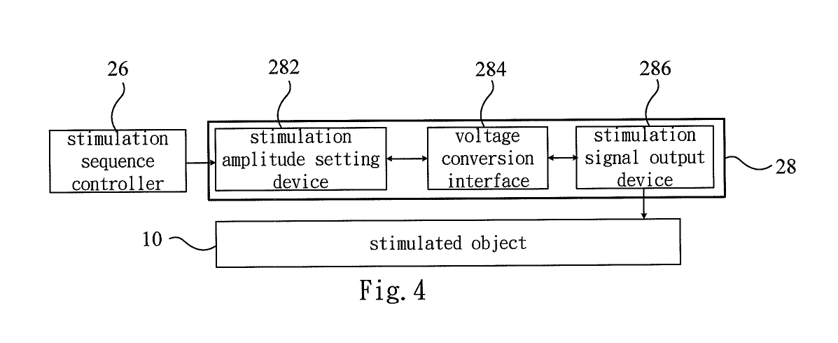

Then, refer to FIG. 4 for block diagram of a stimulation amplitude controller according to the present invention. As shown in FIG. 4, the stimulation amplitude controller 28 includes: a stimulation amplitude setting device 282, which is connected to the stimulation sequence controller 26, and it receives the stimulation clock pulse signal and the stimulation parameters for setting a stimulation amplitude based on the stimulation parameters; a voltage conversion interface 284, connected to the stimulation amplitude setting device 282, and it converts low output voltage to high output voltage; and a stimulation signal output device 286, connected to the voltage conversion interface 284 and is in contact with the stimulated object 10, such that the stimulation amplitude setting device 282 will drive the stimulation signal output device 286 with the high voltage or large current obtained through the voltage conversion interface 284 as based on the stimulation amplitude and the stimulation clock pulse signal, in outputting stimulation pulse signals having the stimulation time, stimulation period, and stimulation amplitude. As such, through the design of voltage conversion interface 284, the stimulation amplitude controller 28 is able to regulate the stimulation voltage based on the operation voltage required for the stimulated object 10.

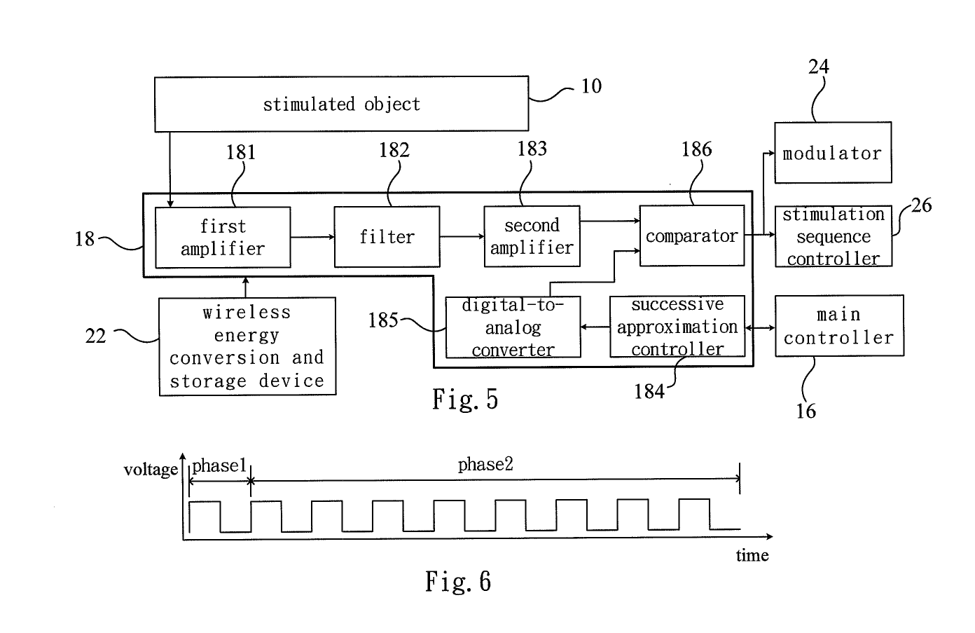

Finally, refer to FIGS. 5 and 6 respectively for a block diagram of a front end sensor according to the present invention, and a waveform diagram of a converted clock pulse according to the present invention. As shown in FIG. 5, the front end sensor 18 comprises: a first amplifier 181, connected to the stimulated object 10, and it receives the physiological signal from the stimulated object, and after amplification, it outputs a first amplified physiological signal; a filter 182, connected between the first amplifier 181 and the second amplifier 183, it receives the first amplified physiological signal, and outputs it to the second amplifier 183 after filtering the signal to within the selected frequency bandwith; a second amplifier 183, connected to the filter 182 for receiving the filtered signal, and it outputs a second amplified physiological signal after a second amplification; and a successive approximation controller 184, connected to the main controller 16, it presets a conversion clock pulse and receives the stimulation parameters to proceed with processing the stimulation parameters according to the conversion clock pulse, and then it outputs a control digital signal; and a digital-to-analog converter 185, connected to the successive approximation controller 184, and it receives the control digital signal and converts it to an analog signal; and a comparator 186, connected to the second amplifier 183, the digital-to-analog converter 185, the stimulation sequence controller 26, and the modulator 24, such that it receives the analog signal and the second amplified physiological signal, and after comparison, it outputs a comparison digital signal, for serving as a result provided to the stimulation sequence controller 26, or as the digital codes provided to the modulator 24.

The conversion clock pulse mentioned above can be classified into having a first phase and a second phase, and in case that the successive approximation controller 184 processing the stimulation parameters based on the conversion clock pulse of the first phase, then the analog signal serves as the sensing threshold value, and the comparison digital signal serves as a result provided to the stimulation sequence controller 26. Or, alternatively, in case that the successive approximation controller 184 processing the stimulation parameters based on the conversion clock pulse of the second phase, then the comparison digital serves as the digital codes provided to the modulator 24.

In the front end sensor 18, the filter 182 can be omitted, so that the physiological signals can be amplified in sequence directly through the first and second amplifiers 181 and 183, and it can just the same provide the result to the stimulation sequence controller 26, or provide the comparison digital signal to the modulator 24.

In the following, the operations of the implantable micro-stimulation device are described, refer to FIGS. 1 and 2. Firstly, the rectifier 222 receives the wireless control signal through the wireless receiver 12 to rectify it into a direct current (DC) voltage and supply it to the power detector 223, which uses the DC voltage to detect the power of the power storage device 221. In case its power is greater than or equal to the power detection value, then the power detector outputs a power supply signal; or in case its power is less that the power detection value, then it outputs a power storage signal. If the power detector 223 outputs a power supply signal, then the power switching device 225 receives the signal, and it will turn on the power output channel of the power storage device 221, which will output its power to a voltage stabilizer 226 for converting it into a stabilized voltage and providing it to the charge pump 227, and the charge pump 227 will convert the stabilized voltage into an operation voltage for providing it to the demodulator circuit 14, the modulator 24, the main controller 16, the front end sensor 18, and the stimulation generator 20 to perform the operations as required.

Alternatively, in case the power detector 223 outputs a power storage signal, then the power switching device 225 receives the signal, and it will turn off the power output channel of the power storage device 221, meanwhile the power supplier 224 also receives the power storage signal and the DC voltage, thus it will perform charging of the power storage device 221. In other words, through the application of the wireless energy conversion and storage interface 22, not only painful surgery operations required for replacing the power storage device in a human body can be avoided, but it can also ensure continued operation of the entire implantable closed-loop micro-stimulation device.

After the demodulator circuit 14, the modulator 24, the main controller 16, the front end sensor 18, and stimulation generator 20 all obtain the power required, refer to FIGS. 1 and 3 for further explanation. The 1-bit comparator 142 receives the wireless control signal and quantifies it into a square wave signal for receiving by the phase-locked loop 144. Then the phase-locked loop 144 outputs a delay signal based on the square wave signal, and transmits it to the phase detector 146. The phase detector 146 determines the phase of the square wave signal according to the delay signal, and generates a result signal based on the phase determined. Finally, a data and clock pulse decoder 148 receives the result signal and the square wave signal, demodulates them into control data and control clock for providing them to the main controller 16.

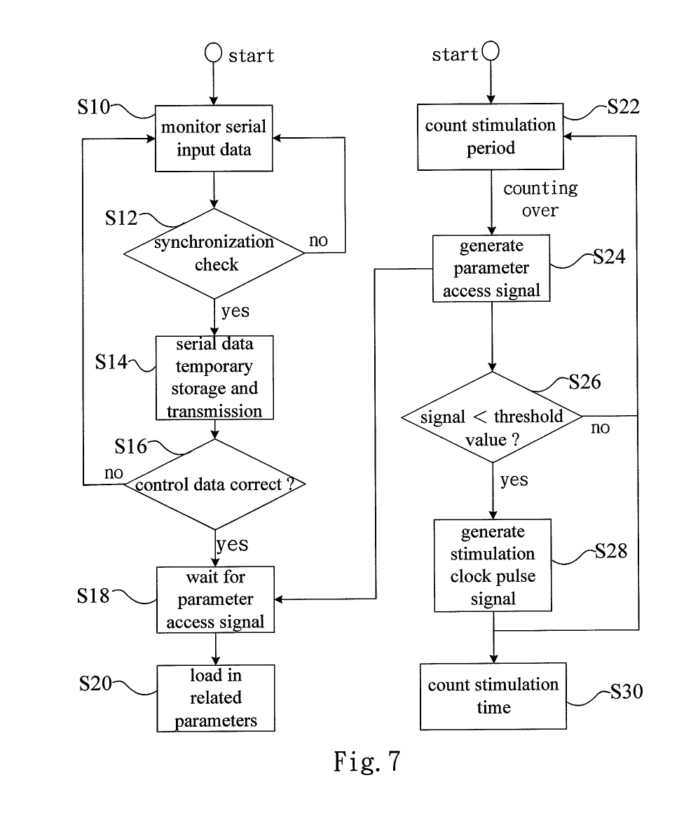

Subsequently, refer to FIGS. 1 and 7. FIG. 7 relates to flowcharts of the steps of operations of a main controller and a stimulation sequence controller respectively according to the present invention. As shown in FIG. 7, firstly, as shown in step S10, the main controller 16 monitors the input control data and control clock. Then, as shown in step S12, based on a set of synchronism values, the main controller 16 determines if the control data and the set of synchronism values are in synchronism. If the answer is negative, then it returns to step S10; otherwise, it starts reading the control data and then executes step S14 to perform temporary storage of data. The end of the control data is provided with a termination code and an error code, thus as shown in step S16, the main controller 16 detects the termination code and the error code of the control data based on the termination detection value and error detection value, in determining if the control data is correct, and if the answer is negative, then it returns to step S10; otherwise, as shown in step S18, the main controller 16 will wait for the access signal. In the present invention, the main controller 16 is provided with detection mechanism, thus it is capable of achieving error detection and data protection during data transmission.

In the present invention, the main controller 16 operates in synchronism with the stimulation sequence controller 26, in the following, the operation flow of the stimulation sequence controller 26 will be described in detail.

Firstly, as shown in step S22, the stimulation sequence controller 26 starts counting the stimulation period, since at this time, none of the stimulation parameters have been loaded, therefore the counting number is zero. Then, as shown in step S24, the stimulation sequence controller 26 outputs an access signal to the main controller 16, so that the main controller 16 may proceed with step S20, namely, generating stimulation parameters and loading them into the front end sensor 18, the stimulation sequence controller 26, and the stimulation amplitude controller 28.

Due to the reloading of the stimulation parameters, the stimulation sequence controller 26 sets the stimulation period and stimulation time based on the reloaded stimulation parameters, and restarts from step S22, namely, starts counting the stimulation period. When the counting is over, it proceeds into step S24 in generating an access signal and transmitting it to the main controller 16. Then, the stimulation sequence controller 26 will execute step S26 based on the result transmitted from the front end sensor 18, in determining if the value of the physiological signal is less than the sensing threshold value, and if the answer is negative, then it returns to step S22; otherwise, it will execute steps S28 and S30 in sequence, and starts generating stimulation clock pulse signal, until the stimulation time counting is over.

Then, refer again to FIG. 4 for a block diagram of a stimulation amplitude controller according to the present invention. As shown in FIG. 4, the stimulation amplitude setting device 282 receives the stimulation parameters and stimulation clock pulse signal via the stimulation sequence controller 26, then it sets the stimulation amplitude based on the stimulation parameters, and it will drive the stimulation signal output device 286 with high voltage or large current obtained through the voltage conversion interface 284 as based on the stimulation amplitude and stimulation clock pulse signal, in outputting a stimulation pulse signal having stimulation time, stimulation period and stimulation amplitude, and applying it on the stimulated object 10.

Finally, refer again to FIGS. 5 and 6. As shown in FIG. 5, the first amplifier 181, the filter 182, and the second amplifier 183 perform the primary amplification, filtering, the secondary amplification for the physiological signal output by the stimulated object 10, in generating a second amplified physiological signal. Then, after the reloading of the stimulation parameters, the successive approximation controller 184 will process the stimulation parameters based on the conversion clock pulse, and will output a control digital signal. Subsequently, the digital-to-analog converter 185 receives the control digital signal and converts it into an analog signal. Finally, the comparator 186 receives the second amplified physiological signal and the analog signal, and after comparison, it will output a comparison digital signal. Since the conversion clock pulse is provided with a first phase and a second phase, therefore, when the successive approximation controller 184 processes the stimulation parameters based on the conversion clock pulse of the first phase, then the analog signal is the sensing threshold value, and the comparison digital signal is the comparison result of the physiological signal and the sensing threshold value, for reading by the stimulation sequence controller 26, and further generating a stimulation clock pulse signal. Or, alternatively, when the successive approximation controller 184 processes the stimulation parameters based on the conversion clock pulse of the second phase, then the comparison digital signal is the digital code, for receiving by the modulator 24 to perform the modulation as required. In other words, the front end sensor 18 may perform feedback processing for the physiological signals, so that the stimulation sequence controller 26 may determine at the start whether it is required to send urgent signals to the stimulated object 10.

In the present invention, a successive approximation controller 184, a digital-to-analog converter 185, and a comparator 186 are utilized to form an analog-to-digital converter having real-time error detection and real-time analog-to-digital conversion capabilities at the same time. Compared with the prior art, the three elements utilized can enable the micro-stimulation device to save the hardware area occupied by two sets of comparators and digital-to-analog converters, thus reducing cost significantly.

Summing up the above, through the application of the present invention, error detection and data protection during data transmission can be achieved, while avoiding the painful surgery operations required for replacing battery in the body of a patient, thus reducing area occupied by the circuit, and fully fulfilling requirements of an implantable micro-stimulation device.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. An implantable closed-loop micro-stimulation device, used to contact a stimulated object, comprising:
a wireless receiver receiving a wireless control signal;
a demodulator circuit, connected to said wireless receiver to receive said wireless control signal, and demodulates it into control data and a control clock;
a main controller, connected to said demodulator circuit, and receives said control data and said control clock, and detects a termination code and an error code of said control data based on preset termination detection value and error detection value, in determining correctness of said control data, and when said control data is correct, it generates a plurality of stimulation parameters based on said control data and said control clock;
a front end sensor, connected to said main controller and said stimulated object, and receives said stimulation parameters in generating a sensing threshold value, said front end sensor receives physiological signals transmitted from said stimulated object and compares them with said sensing threshold value and then outputs comparison results; and
a stimulation generator, connected to said front end sensor and said main controller and is in contact with said stimulated object, said stimulation generator works in synchronism with said main controller, and outputs an access signal to said main controller for generating said stimulation parameters, then said stimulation generator receives said stimulation parameters and said comparison results, and when said physiological signals is less than said sensing threshold value, it generates a stimulation pulse signal based on said stimulation parameters, to be applied on said stimulated objects.

2. The implantable closed-loop micro-stimulation device as claimed in claim 1, further comprising: a wireless energy conversion and storage interface, connected to said wireless receiver, said demodulator circuit, said main controller, said front end sensor, and said stimulation generator, and receives said wireless control signal and converts it to an operation voltage through a power management mechanism, for supplying it to said demodulator circuit, said main controller, said front end sensor, and said stimulation generator to perform operations as required.

3. The implantable closed-loop micro-stimulation device as claimed in claim 2, wherein said wireless energy conversion and storage interface further comprising:
a power storage device;
a rectifier, connected to said wireless receiver to receive said wireless control signal, for rectifying it into a direct current (DC) voltage;
a power detector, connected to said rectifier and said power storage device, and it presets a power detection value, said power detector receives said DC voltage to detect power of said power storage device, and when said power is greater than or equal to said power detection value, it outputs a power supply signal, and when said power is less than said power detection value, it outputs a power storage signal;
a power supplier, connected to said rectifier and said power detector, it receives said power storage signal and said DC voltage for charging said power storage device;
a power switching device, connected to said power detector and said power storage device, it receives said power supply signal or said power storage signal, to selectively turn on or turn off a power output channel of said power storage device;
a voltage stabilizer, connected to said power switching device, and receives power through said power output channel of said power storage device, and converts it into a stabilized voltage; and
a charge pump, connected to said voltage stabilizer, said demodulator circuit, said main controller, said front end sensor, said stimulation generator, and it receives said stabilized voltage and converts it into said operation voltage.

4. The implantable closed-loop micro-stimulation device as claimed in claim 1, wherein said demodulator circuit further comprising:
a 1-bit comparator, connected to said wireless receiver, and it receives said wireless control signal, and quantifies it into a square wave signal;
a phase-locked loop, connected to said 1-bit comparator, and receives said square wave signal for outputting a delay signal;
a phase detector, connected to said phase-locked loop, and receives said delay signal, so as to determine phase of said square wave signal, then generates a result signal based on said phase; and
a data and clock decoder, connected to said phase detector, said 1-bit comparator, said main controller, to receive said result signal and said square wave signal for demodulating them into said control data and said control clock.

5. The implantable closed-loop micro-stimulation device as claimed in claim 1, further comprising: a modulator, connected to said front end sensor, which receives said physiological signal, and converts it into a digital code and then transmits it into said modulator, for modulating and then outputting said modulating signal.

6. The implantable closed-loop micro-stimulation device as claimed in claim 1, wherein said main controller presets a set of synchronism values, said main controller first determines whether said control data and said set of synchronism values are in synchronism based on said set of synchronism values, then it determines whether said control data is correct.

7. The implantable closed-loop micro-stimulation device as claimed in claim 1, wherein said stimulation generator further comprising:
a stimulation sequence controller, connected to said front end sensor and said main controller, said stimulation sequence controller operates in synchronism with said main controller, and it outputs said access signal to said main controller, for it to generate real-time said stimulation parameters as based on said access signal, said stimulation sequence controller receives said stimulation parameters and said result, and it sets a stimulation time and a stimulation period as based on said stimulation parameters, and when said physiological signal is less than said sensing threshold value, it generates a stimulation clock pulse signal having said stimulation time and said stimulation period; and
a stimulation amplitude controller, connected to said stimulation sequence controller, and is in contact with said stimulated object, said stimulation amplitude controller receives said stimulation clock pulse signal and said stimulation parameters, and it sets a stimulation amplitude based on said stimulation parameters, and outputs said stimulation pulse signal having said stimulation time, said stimulation period, and said stimulation amplitude.

8. The implantable closed-loop micro-stimulation device as claimed in claim 7, wherein said stimulation amplitude controller further comprising:
a stimulation amplitude setting device, connected to said stimulation sequence controller, and receives said stimulation clock pulse signal and said stimulation parameters, and it sets said stimulation amplitude based on said stimulation parameters;
a voltage conversion interface, connected to said stimulation amplitude setting device, and it converts low voltage output into high voltage output; and
a stimulation signal output device, connected to said voltage conversion interface, and is in contact with said stimulated object, said stimulation amplitude setting device drives said stimulation signal output device with high voltage or high current obtained through said voltage conversion interface as based on said stimulation amplitude and said stimulation clock pulse signal, into outputting said stimulation pulse signal having said stimulation time, said stimulation period, and said stimulation amplitude.

9. The implantable closed-loop micro-stimulation device as claimed in claim 1, wherein said front end sensor further comprising:
a first amplifier, connected to said stimulation object, and it receives and amplifies said physiological signal, and then outputs a first amplified physiological signal;
a second amplifier, connected to said first amplifier, and it receives and amplifies said first amplified physiological signal, and then outputs a second amplified physiological signal;
a successive approximation controller, connected to said main controller, and it presets a conversion clock pulse, said successive approximation controller receives said stimulation parameters, and it processes said stimulation parameters based on said conversion clock pulse for outputting a control digital signal;
a digital-to-analog converter, connected to said successive approximation controller, and it receives said control digital signal, and converts it into an analog signal; and
a comparator, connected to said second amplifier, said digital-to-analog converter, and said stimulation generator, and it receives said second amplified physiological signal and said analog signal, and after comparison, it outputs a comparison digital signal serving as a result or a digital code.

10. The implantable closed-loop micro-stimulation device as claimed in claim 9, wherein said conversion clock pulse has a first phase and a second phase, such that when said successive approximation controller processes said stimulation parameters based on said first phase of said conversion clock pulse, said analog signal is said sensing threshold value, and said comparison digital signal serves as said result provided to said stimulation generator; and when said successive approximation controller processes said stimulation parameters based on said second phase of said conversion clock pulse, said comparison digital signal serves as said digital code.

11. The implantable closed-loop micro-stimulation device as claimed in claim 9, wherein said front end sensor further comprises a filter, connected to said first and second amplifiers, and it receives said first amplified physiological signal, and filters it to obtain said first amplified physiological signal within preset bandwidth, and it outputs said signal to said second amplifier.
